(19) United States
(12) Patent Application Publication
(10) Pub. No.: US 2007/0065828 A1
(43) Pub. Date: Mar. 22, 2007

(54) Oligonucleotide for genotyping of mycoplasma, microarray comprising the oligonucleotide, and method for detection of species using the microarray

(75) Inventors: Cheol-Min Kim, Busan-city (KR); Hee-Kyung Park, Seoul (KR); Hyun-Jung Jang, Busan-city (KR); Hyo-Myoung Kim, Busan-city (KR)

Correspondence Address: THE RAFFERTY PATENT LAW FIRM, 5641 BURKE CENTRE PKWY, SUITE 100, BURKE, VA, 22015-1731, US

(73) Assignee: GENEIN CO., LTD., Busan-city (KR)

(21) Appl. No.: 10/597,173

(22) PCT Filed: Jan. 14, 2005
(86) PCT No.: PCT/KR05/00147

§ 371 Date: Jul. 13, 2006

(30) Foreign Application Priority Data
Jan. 14, 2004 (KR) 2004-0002715

(51) Int. Cl. C12Q 1/68; C12M 3/00

(52) U.S. Cl. 435/6; 435/287.2; 977/924; 257/E51.02

(57) ABSTRACT

The present invention relates to a method for detecting Mycoplasma and its related strains which are source of contamination of cell lines and biological products and human pathogenic. More particularly, the present invention relates to genus-specific and species-specific oligonucleotides for genotyping of Mycoplasma, Acholeplasm and Ureaplasma strains, microarray comprising the oligonucleotides, and method for detection of species using the microarray.

As described above, the present invention provides a rapid and accurate assay method capable of simultaneously detecting many Mycoplasma and its related strains from a single sample using a microarray comprising novel oligonucleotides for detecting Mycoplasma and its related strains which are known as a source of contamination of cell lines and biological products and human pathogenic. Further, the present invention provides an objective and credible assay method capable of tracing a contamination source for preventing expansion of infective Mycoplasma and its related strains and controlling a contamination of Mycoplasma against biological products and stem cells or cord blood cells which are useful for gene therapy and cell therapy.

### TECHNICAL FIELD

The present invention relates to a method for detecting Mycoplasma and its related strains which are a source of contamination of cell lines and biological products and human pathogens. More particularly, the present invention relates to genus-specific and species-specific oligonucleotides for genotyping Mycoplasma, Acholeplasm and Ureaplasma strains, a microarray comprising the oligonucleotides, and a method for detecting strains using the microarray.

### BACKGROUND ART

Mycoplasma is a prokaryote pertaining to Mollicute family without cell wall, which was known as a hospital acquired pathogen causing pneumonia via infection of genital and respiratory organs of human as well as livestock such as pig and cow. Recently, Mycoplasma is more seriously understood as a major contaminant of cell culture and cell line.

Especially, as the development and production of biological products for protecting and treating human diseases increases, the contamination of various pathogens provided by microorganism or clinical sample in the process of production became a serious problem. Examples of the biological products are an oncolytic virus, vaccine, a gene therapy vector and a recombinant protein. They have been found to be contaminated by bacteria, fungus, virus, Mycoplasma and its related strains (Doblhoff-Dier et al., 2001). The reason of the contamination is an organism contaminated in media components or experimental instruments and cross-contamination of microorganism and virus in air (Jung et al., 2003). Also, the contamination can be occurred by a cross-contamination of already-infected WCB (Working Cell Bank) which is used for mass production of biological products (Wisher et al., 2002).

It is reported that, among these contamination sources, about 15-35% of cell culture or cell line is infected by Mycoplasma and its related strains (Hopert et al., 1993). This also makes experimental results incredible because it can change characteristics of cells such as abnormal synthesis of DNA, RNA and protein by binding to host cell wall (Kong et al., 2001). As gene therapy and cell therapy are getting into the spotlight recently, an assay for infection of stem cell and cord blood by Mycoplasma and its related strain became more important. Therefore, for the credible and reproducible experimental results and the quality control of commercialized biological products, it is essential to detect an infection with Mytcoplasma and its related strains.

Under this situation, Europe community make it a rule that, for credibility of safety and quality of food and drug, GMP (Good Manufacturing Practice) and QC (Quality Control) should be submitted and cell banks such as MCB (Master Cell Bank) and WCB should be subjected to an assay for detection of virus, fungus and bacteria such as Mycoplasma (Doblhoff-Dier et al., 2001).

About 100 kinds of bacteria pertaining to Mollicute family without cell wall have been found so far, including Acholeplasma, Enteroplasma, Mesoplasma, Mycoplasma, Ureaplasma and Spiroplasma. Among them, about 20 kinds of Mycoplasma, Acholeplasma and Ureaplasma are major contamination source of cell culture. These are referred to as “Mycoplasma and its related strains” in this specification. About 95% of the contaminants are covered by M. arginini, M. fermentans, M. orale, M. hyorhinis, M. hominis, M. salivarium, M. pirum, A. laidlawii (Dorigo-zetsma et al., 1997). However, Mycoplasma is difficult to be cultured in extracellular media and turbidity is rare in the culture. Therefore, there has been a need to the rapid and accurate genotypic detection method which can trace a contamination source of Mycoplasma and its related strains.

Conventional Mycoplasma detection methods are the culturing method, the DNA fluorochome stain method, the immunofluorescence method, and the polymerase chain reaction (PCR) method (Dorigo-zetsma et al., 1997). However, the culturing method has a drawback that extracellular culturing is difficult, preparing its media is complex by adding supplements such as serum and culturing time is too long, about 4 days˜3 weeks according to the kinds of strains (Jensen et al., 2003). The DNA fluorochome stain method such as Hoechest 33258 stain has a drawback that culturing condition is too difficult to match and subjective inspectors can make a misjudgment (Chen et al., 1997). The immunofluorescence method such as ELISA has a drawback that bacteria having similar antigen with Mycoplasma such as Streptococcus milleri group and Staphylococcus aureus may raise a false positive signal due to of low specificity (Hopert et al., 1993). The PCR method makes use of 16S/23S intergenic spacer region (ITS) and a gene coding 169 kDa of P1 cyadhesion proteine which represent variety of Mycoplasma (Uphoff et al., 2002). The P1 gene, a surface antigen gene, has several subtypes representing diversity and has been used as a target gene for serological detection using immune reaction and genotypic detection using restriction fragment length polymorphism (RFLP) to identify Mycoplasma (Campo et al., 1998). However, most of conventional PCR methods use a primer designed based on 16S rRNA which is a common sequence of prokaryotes, and second PCR or nested PCR having high sensitivity can make a cross-contamination of Mycoplasma dispersed in air and an amplification of a bacteria similar with Mycoplasma in classification (Uphoff et al., 2002).

To overcome the above limitations of the conventional detection methods, a genotypic detection method using probes have been developed recently, which make it possible to analyze many kinds of genes in a short time using DNA hybridization principle based on gene sequencing and detect specifically a single base change using a proper hybridization condition between specific probe and target DNA.

The present inventors developed ITS-derived oligonucleotides capable of detecting Mycoplasma and its related strains, which are important in genotypic detection, and a mycroarray comprising the oligonucleotides as a probe for detecting Mycoplasma and its related strains.

### DISCLOSURE OF THE INVENTION

It is a first object of the present invention to provide oligonucleotides for detecting Mycoplasma and its related strains designed based on their ITS base sequences.

It is another object of the present invention to provide novel ITS sequences of Mycoplasma bovis, Mycoplasma cloacale, Mycoplasma falconis, Mycoplasma faucium, Mycoplasma spermatophilum and Mycplasma synoviae, which is useful for detecting Mycoplasma and its related strains.

It is another object of the present invention to provide a microarray comprising genus-specific and species-specific oligonucleotides for detecting Mycoplasma and its related strains as probes.

It is another object of the present invention to provide a method for detecting Mycoplasma and its related strains using the microarray.

It is another object of the present invention to provide a kit for diagnosing Mycoplasma and its related species infection individually or simultaneously, comprising genus-specific and species-specific oligonucleotides for genotyping Acholeplasma and related strains.

According to an aspect of the present invention, there is provided a purified ITS (internal transcribed spacer) target DNA for genotyping Mycoplasma strains, comprising any one sequence selected from SEQ ID Nos. 1 to 6.

SEQ ID Nos. 1 to 6 are base sequences of ITS (internal transcribed spacer) of Mycoplasma bovis, Mycoplasma cloacale, Mycoplasma falconis, Mycoplasma faucium, Mycoplasma spermatophilum and Mycplasma synoviae, which was newly obtained by base sequencing analysis.

The ITS target DNA of the present invention can be used indirectly for designing probes or primers used for genotyping Mycoplasma strains or directly for genotyping Mycoplasma strains via PCR amplification.

According to another aspect of the present invention, there is provided an oligonucleotide for genus-specific genotyping of Mycoplasma and Ureaplasma strains, comprising any one sequence selected from SEQ ID Nos. 7 to 21 or its complementary sequence.

According to another aspect of the present invention, there is provided an oligonucleotide for genus-specific genotyping of Acholeplasma strains, comprising any one sequence selected from SEQ ID Nos. 22 to 27 or its complementary sequence.

According to another aspect of the present invention, there is provided an oligonucleotide for species-specific genotyping of Mycoplasma and Ureaplasma strains, comprising any one sequence selected from SEQ ID Nos. 28 to 127 or its complementary sequence.

According to another aspect of the present invention, there is provided an oligonucleotide for species-specific genotyping of Acholeplasma strains, comprising any one sequence selected from SEQ ID Nos. 128 to 133 or its complementary sequence.

The oligonucleotides according to the present invention are designed based on multiple sequence alignment of ITS (internal transcribed spacer) sequences, which are present between 16S rRNA and 23S rRNA of Mycoplasma and its related species. The oligonucleotides can be used as primers for PCR amplification in order to genotype Mycoplasma and its related species or as probes for hybridization reaction in order to genotype Mycoplasma and its related species.

According to another aspect of the present invention, there is provided a microarray comprising more than one oligonucleotides selected from genus-specific and species-specific oligonucleotides for genotyping Mycoplasma, Acholeplasma and Ureaplasma strains according to any one from claims 2 to 5 as probes attached on a support.

In the microarray according to the present invention, the probes may be any materials having base sequence, preferably any one selected from a group consisting of DNA (Deoxyribose Nucleic acid), RNA (Ribose Nucleic Acid), and nucleic acid analogues such as PNA (Peptide Nucleic Acid), LNA (Locked Nucleic Acid) and HNA (Hexitol Nucleic Acid).

In the microarray according to the present invention, the support may be any materials to which the probes can be attached, preferably any one selected from a group consisting of slide glass, plastic, membrane, semiconductive chip, silicon and gel. The microarray according to the present invention can be manufactured using conventional method such as pin microarray, ink jet, photolithography or electric array method.

The microarray according to the present invention can be used for simultaneously genotyping various Mycoplasma and its related species which are known as a major contaminant of biological drug and cell line as well as a human pathogen from one sample, as the microarray comprises genus-specific and species-specific oligonucleotides for genotyping Mycoplasma and its related species as a set attached a support.

According to another aspect of the present invention, there is provided a method for detecting Mycoplasma, Acholeplasma and Ureaplasma strains, comprising the following steps:

a) extracting nucleic acids from a sample;

b) amplifying target DNA among the extracted nucleic acids;

c) hybridizing the amplified target DNA with probes of the microarray according to the above present invention; and

d) detecting signals generated from the hybridization reaction.

In the detection method according to the present invention, the sample may be biological drug, cell line, or human tissues or serum. The purifying step can be performed using conventional DNA or RNA purification method or kit. The signal detecting step can be performed using a conventional fluorescence scanner after binding conventional fluorescent dyes such as Cy5 or Cy3.

According to another aspect of the present invention, there is provided a kit for diagnosing Mycoplasma and its related species infection, comprising more than one oligonucleotide selected from genus-specific and species-specific oligonucleotides for genotyping Acholeplasma, Mycoplasma and Ureaplasma strains according to the above present invention.

In the kit according to the present invention, the oligonucleotides are used as probes for hybridizing with target sample and may be contained in a proper vessel. The probes may be labeled with a radioactive or non-radioactive labeling agent, the latter comprises conventional biotin, Dig(digoxigenin), FRET(fluorescence resonance energy transfer) or fluorescent dye (Cy5 or Cy3). Further, the oligonucleotides can be used as primers for PCR amplification. In this case, the kit may contain DNA polymerase, 4 dNTPs and PCR buffer for PCR reaction. In addition, the oligonucleotides can be attached to a microarray as probes. In this case, the kit may contain hybridization reaction buffer, PCR kit containing primers for amplifying a target gene, washing solution for the unhybridized DNA, dyes, washing solution for unbound dyes and manual sheet for the microarray.

Hereafter, the present invention will be described in more detail.

The present invention provides a method for detecting or genotyping Mycoplasma and its related strains which is a major contamination source of cell lines and biological products and a human pathogen, comprising the following steps:

a) if necessary, extracting nucleic acids from a sample such as cell lines, biological products or human tissue or serum;

b) if necessary, amplifying target DNA of Acholeplasma, Mycoplasma and Ureaplasma strains among the extracted nucleic acids using more than one proper primers;

c) hybridizing the amplified target DNA with probes having a sense or antisense or complementary sequences of genus-specific and species-specific oligonucleotides of Acholeplasma, Mycoplasma and Ureaplasma strains disclosed in Tables 2 and 3; and

d) detecting signals generated from the hybridization reaction.

From the detected signals in the step d), the existence of Mycoplasma and its related strains in the sample can be predicted.

The present inventors carried out a sequence analysis of ITS regions of many Acholeplasma, Mycoplasma and Ureaplasma strains to obtain genus-specific and species-specific oligonucleotides for detecting Mycoplasma and its related stains which can be a basis of developing a specific and sensitive hybridization assay. Also, the present inventors newly analyzed ITS sequences of newly found 6 Mycoplasma strains, which makes it possible to design probes capable of detecting more various Mycoplasma and its related strains.

Table 1 discloses ITS sequences of newly analyzed 6 strains among target sequences for detecting Mycoplasma strains, which correspond to SEQ ID Nos. 1 to 6. In the present invention, the probes for detecting Mycoplasma strains were designed based on the multiple alignment of ITS sequences of Mycoplasma.

FIGS. 1 and 2 show multiple sequence alignments of ITS regions of Mycoplasma, Acholeplasma and Ureaplasma for selecting genus-specific and species-specific probes of Mycoplasma and its related strains. Genus-specific oligonucleotides of Mycoplasma and Ureaplasma were designed from conservative sequence region indicated by a box in FIGS. 1a to 1f. Species-specific oligonucleotides of Mycoplasma and Ureaplasma were designed from polymorphic sequence region outside the box in FIGS. 1a to 1f. Genus-specific oligonucleotides of Acholeplasma were designed from conservative sequence region indicated by a box in FIGS. 2a to 2c. Species-specific oligonucleotides of Acholeplasma were designed from polymorphic sequence region outside the box in FIGS. 2a to 2c.

In step b) of the present invention, the target DNA of Acholeplasma, Mycoplasma and Ureaplasma strains were amplified using more than one pair of proper primers. FIG. 3 shows PCR amplification of ITS target sequences of Mycoplasma and its related strains using a primer pair, MP16SF-2 and MP23SR-2. In FIG. 3, 1 is a PCR product of M. arginini, 2 is a PCR product of M. arthritidis, 3 is a PCR product of M. fermentans, 4 is a PCR product of M. hominis, 5 is a PCR product of M. hyorhinis, 6 is a PCR product of M. neurolyticum, 7 is a PCR product of M. opalescens, 8 is a PCR product of M. orale, 9 is a PCR product of M. pirum, 10 is a PCR product of M. penetrans, 11 is a PCR product of M. pulmonis, 12 is a PCR product of M. salivarium, 13 is a PCR product of M. cloacale, 14 is a PCR product of M. falconis, 15 is a PCR product of M. faucium, 16 is a PCR product of M. hyosynoviae, 17 is a PCR product of M. muris, 18 is a PCR product of M. primatum, 19 is a PCR product of M. spermatophilum, 20 is a PCR product of M. synoviae, 21 is a PCR product of M. pneumoniae, 22 is a PCR product of M. genitalium, 23 is a PCR product of M. bovis, 24 is a PCR product of U. urealyticum, 25 is a PCR product of A. laidlawii.

In step c) of the present invention, the amplified target DNA were hybridized with probes for detecting Mycoplasma and its related strains. Preferably, the probes may be a combination of more than one probes capable of simultaneously detecting many Mycoplasma and its related strains from a single sample. Practically, the probes are optimized to simultaneously hybridize with multiple target DNAs of Mycoplasma and its related strains under the same hybridization and washing conditions.

The present invention provides a microarray comprising a set of probes for detecting Mycoplasma and its related strains, which can simultaneously detect many Mycoplasma and its related strains from a single sample with a single experiment.

In the present invention, the term ‘probe’ means a single-stranded oligonucleotide having a sequence complementary to target DNA of Mycoplasma, Acholeplasma and Ureaplasma. The probe may have a sense, antisense or complementary sequence of SEQ ID Nos. disclosed in this specification as long as it can hybridize with one of double strands of target DNA. The oligonucleotide may be ribonucleotide (RNA), deoxynucleotide (DNA), peptide nucleic acid (PNA) or locked nucleic acid (LNA), and contain modified nucleotides such as Inosine only if it does not change their hybridization characteristics. Preferably, the genus-specific oligonucleotides for detecting Mycoplasma, Acholeplasma and Ureaplasma may have a base sequence of SEQ ID Nos. 7 to 27.

Preferably, the species-specific oligonucleotides for detecting Mycoplasma, Acholeplasma and Ureaplasma may have a base sequence of SEQ ID Nos. 28 to 133.

FIG. 4 shows a microarray comprising probes for detecting genotypes of Mycoplasma and its related strains as a set on a support. In FIG. 4, each species name and SEQ IN Nos. are described which correspond to individual probes. The terms ‘MP-C’ and ‘AP-C’ mean Mycoplasma and Ureaplasma genus and Acholeplasma genus. FIG. 4 is no more than an example of probe compartment of the present invention, so compartment and layout of each probe can be varied.

In the present invention, newly analyzed ITS sequences of 6 Mycoplasma strains as a target DNA for detecting Mycoplasma and its related strains are as shown in Table 1. The genus-specific oligonucleotides for detecting Mycoplasma, Acholeplasma and Ureaplasma used in the present invention are as shown in Table 2. The species-specific oligonucleotides for detecting Mycoplasma, Acholeplasma and Ureaplasma used in the present invention are as shown in Table 3.

TABLE 1

| | | SEQ |
|---|---|---|
| | | ID |
| Species | Sequence (5′ → 3′) | NO |
| M. bovis | TTCTACGGAGTACACTTGTCTTTTATCACTAT | 1 |
| | AAAAAAAAGACTTATAACCAAAATTACTAGAC | |
| | CTATATTTATTTATAAACGTCATGGCTTTTAT | |
| | TAATAGGTCAAAAGCTATATATCTAGTTTTGA | |
| | GAGAACATTCTCTCATATGTTCTTTGAAAACT | |
| | GAATAGTAAAATATTTTTCGATATTTACAACG | |
| | ACATCAAAAATCAAATTAATGGTTAATTTGTT | |
| | TTGATTCATCGAGTAAGTCATATTTAATATGA | |
| | TTCATTGAAATGTCTTAAAATACACATCTAAA | |
| | ACTAACAACAATAGGAAAATACTACTTTTAAA | |
| | TAAGGAAGAGTTTTTGGTGGATGC | |
| M. cloacale | CTTCTACGGAGTACAATTCTCACTGTTATGGA | 2 |
| | ATTAAATTTGTATCCAGTTTTGAGAGAACTTT | |
| | CTCTCAATTTTGTTCTTTGAAAACTGAATATA | |
| | GACATTGAAATCAATAAATTAATATTTCAAAT | |
| | GTTTAGATCAACCTATAGAATATTCAAGACAT | |
| | ATACAAAAATAGGTCATACTTATATTTATAAA | |
| | TACT | |
| M. falconis | CTTTCTACGGAGTACAACTTCTGTTATGGAAT | 3 |
| | AATATTTGTATCCAGTTTTGAGAGTACTAACT | |
| | CTCTTTTTGTTCTTTGAAAACTGAATATCGAC | |
| | ATTGAAAAATTATTAATTAATATTTTCAAAGT | |
| | TTAGATCAACCTATAGAATACAAAAATATAGA | |
| | CAACAATAGGTCATACAACAAACATAACAAAA | |
| | CAACT | |
| M. faucium | GAATGGTGGCTTCGAGACTAAAAGTTATGGAA | 4 |
| | AAACATCGTATCCAGTTTTGAGAGAACTAAAC | |
| | TTCTCTCTTTTGTTCTTTGAAAACTGAATATA | |
| | GACATTGAAAATTAAAAAATTAATATTTCAAA | |
| | GTTTAGATCAACCTATAGAATACAAAATCAAT | |
| | ACAATAGGTCAATACTATACAATTGCATAACA | |
| | AAAAATACTATTAAACAAGATAAGAGTTTTTG | |
| | GTGGATGCAATTGTA | |
| M. | GTGGGGATGGATCACCTCCTTTCTACGGAGTA | 5 |
| spermatophilum | CAAACATACATTCAAATTTTGACTGAATGTTA | |
| | TTAACCTTATTTTTTCACTAGGCCTTTTTAAT | |
| | ATATTTTGTTATGTGACTTTTATGGCCTAAAA | |
| | GTCTTATATCTAGTTTTGAGAGGACATCCTCT | |
| | CTAATTGTTCTTTGAAAACTGAATAGTAAATT | |
| | TTTTGATATTTACAACGACATCTAAATAATTG | |
| | AATTAAGTCAATTTGTTTAGATTTCATCGAGA | |
| | TAGTCATTTTAAAAAAATGATTCATTGAAATG | |
| | TCTTAAAATACACATCAAAACAAACAATCTAT | |
| | ACAATAGGAATTTATATACT | |
| M. synoviae | TCCTTACGGAGTACATTAATTTTACAAAAGGC | 6 |
| | ATTTTTATTAACTGAAAGCTTTTAGAGAAAAA | |
| | TTCTAAAAGCGGTTGTGTATCGCTTTTTTTGC | |
| | CTTGGGCTATTGTATTTAGTTTTGAGAGAACA | |
| | ACCTCTCTTAAAATTGTTCTTTGAAAACTAAA | |
| | TAGTAATAAAGATATTACAACGACATCAAAAA | |
| | TATAAATTAATTAAGGTTAATTTGTTTTGATA | |
| | CCGAGTTTAAATTATTGAATAATAATTTATTA | |
| | AAATGTCTTTGAATACATCATAACAATATAAC | |
| | AATAGGACATATTGATACTAACTTTTAAAAAA | |
| | GT | |

TABLE 2

| | | | |
|---|---|---|---|
| | | | SEQ |
| | | | ID |
| Genus | Probe | Sequence | NO |

| | | | |
|---|---|---|---|
| Mycoplasma | MP-CP1 | TTCTTTGAAAACTGA | 7 |
| | MP-CP2 | RWTCTTTVAAAACTRRATWN | 8 |
| M. argini, | MP-CA1 | MWTYGTRTCCAGTTTTGAGAG | 9 |
| M. arthritidis | MP-CA2 | TTTAGATCAACCTATAGAATA | 10 |
| M. cloacale, | | | |
| M. falconis | | | |
| M. faucium, | | | |
| M. hominis | | | |
| M. hyosynoviae, | | | |
| M. orale | | | |
| M. salivarium | | | |
| M. bovis, | MP-CB1 | RTATYTAGTTTTGAGAGRRCA | 11 |
| M. fermentans | MP-CB2 | WWTRATTYATTRAAATGTCTT | 12 |
| M. opalescens, | MP-CB3 | GGKYAATTTGTTTWGAT | 13 |
| M. primatum, | MP-CB4 | RATATTTACAMCGMCAYC | 14 |
| M. spermatophilum, | | | |
| M. synoviae | | | |
| M. muris, | MP-CC1 | CCTCCTTTCTATCGGAGTAMA | 15 |
| M. pentrans | MP-CC2 | CGGATTCTATTTAGTTTTGAG | 16 |
| U. urealyticum | | | |
| M. neurolyticum, | MP-CD1 | TAAAATAGATACCTTAAKATA | 17 |
| M. pulmonis | MP-CD2 | GTATYYAGTTTTGAAAG | 18 |
| | MP-CD3 | CTTGCCAAWTAGWTWT | 19 |
| M. genitalium, | MP-CE1 | AWACRACAATCTTTCTAGTTC | 20 |
| M. pirum | MP-CE2 | AATAAGTTACTAAGGGCTTAT | 21 |
| M. penumoniae | | | |
| Acholeplasma | AP-CP1 | TCATCATATTCAGTTTTG | 22 |
| | AP-CA1 | GGGCCTRTAGCTCAGYTGGTT | 23 |
| | AP-CA2 | AGAGCRCWCGCYTGATAAGCG | 24 |
| | AP-CA3 | WGRGGTCGATGGTTCRAGTCC | 25 |
| | AP-CB1 | TCATCATATTCAGTTTTGARR | 26 |
| | AP-CB2 | AGTCTTTGAAAAGTAGATAAA | 27 |

TABLE 3

| | | | |
|---|---|---|---|
| | | | SEQ |
| | | | ID |
| Species | Probe | Sequence | NO. |

| | | | |
|---|---|---|---|
| M. arginini | MP-arg1 | AGATTATATCATACAATAGA | 28 |
| | MP-arg2 | GAGTACATAAATGTTATGGAA | 29 |
| M. | MP-arf1 | TGAAGCCCGATGGTGGCTTCG | 30 |
| arthritidis- | MP-arf2 | TGAGAGAACTAAACTTCTCTC | 31 |
| faucium | MP-arf3 | GAATACAAAATCAATACAATA | 32 |
| M. fermentans | MP-fer1 | ATGTACTATTAACTTATTTCAC | 33 |
| | MP-fer2 | TACAAAAGAGTACTTTTTAAA | 34 |
| | MP-fer3 | TTTTTATGGGTCTAAAGCTTT | 35 |
| | MP-fer4 | GAACAATATTTTTTTCTCTCA | 36 |
| | MP-fer5 | ATAACAAACTATAACAATAGG | 37 |
| M. hominis | MP-hom1 | ATTTATCTCTCGGTTCTTT | 38 |
| | MP-hom2 | ATATTTATATTTTATAAGACA | 39 |
| | MP-hom3 | ATTGATATATTAATTAATATT | 40 |
| M. hyorhinis | MP-hyo1 | GAATAGCAAATAACAATATGATT | 41 |
| | MP-hyo2 | CGGAGTACATTAGTCTTAATT | 42 |
| | MP-hyo3 | TTACATAATCGATTCGTGTCT | 43 |
| | MP-hyo4 | AGCTTTAAGTTCTCAATTATA | 44 |
| | MP-hyo5 | TTCATATTTATTATTTCAACG | 45 |
| | MP-hyo6 | AACGATCTTTTTTATAACCGA | 46 |
| | MP-hyo7 | TTAAATTTCTAAAATAGATTA | 47 |
| | MP-hyo8 | AGATATTTATCTTTAGCAATA | 48 |
| M. | MP-neu1 | GGTTATTATGGGCTTGCTA | 49 |
| neurolyticum | MP-neu2 | GGTTATTTAAAAATCCTTTTA | 50 |
| | MP-neu3 | TAATTTTTTCTTTCTAATTAA | 51 |
| M. opalescens | MP-opa1 | CATCATAATGTAACCAATAC | 52 |
| | MP-opa2 | ACAAAAATCATTATTTTTAAT | 53 |
| | MP-opa3 | TTTAATGATTATTAACCTTTT | 54 |
| | MP-opa4 | TTATGTGCTTTGTTTTTATGG | 55 |
| | MP-opa5 | TATGGTCTACAAAGCTTATAT | 56 |
| | MP-opa6 | GATAAAAAACAATCATAAATT | 57 |
| M. orale | MP-ora1 | CATAAATAGTTAATGGCTCA | 58 |
| | MP-ora2 | ATAGAGACAAATACAAAAACA | 59 |
| | MP-ora3 | GGTCAAAAATACTTATACGTA | 60 |
| M. pirum | MP-pir1 | TAGTTCTTTGTGTGAATAACA | 61 |
| | MP-pir2 | CTTTATACACCTTATTACAAT | 62 |
| | MP-pir3 | TAAAATCCAATTTAAATGTTA | 63 |
| | MP-pir4 | GCAAATTTGATGTCAACATTT | 64 |
| | MP-pir5 | AATTAATCTCTCCTATTACTT | 65 |
| | MP-pir6 | TTAAAGTAGTAGAGATGGTTC | 66 |
| | MP-pir7 | CAAATATCAAATGCTAATGGA | 67 |
| | MP-pir8 | ATGCTAATGGATATCAAAAAA | 68 |
| M. penetrans | MP-pen1 | AAGAGTAAGTTCTAGGTCG | 69 |
| | MP-pen2 | CATTAAAGCTAAGTAACAAAT | 70 |
| | MP-pen3 | TCCTAAACTGAAATTTATCT | 71 |
| | MP-pen4 | TTATATAAGAGTAAGTTCTAG | 72 |
| | MP-pen5 | ATTTTTCTCTCAAGATAGTTC | 73 |
| | MP-pen6 | TCTAATCATACTTGTTATTTT | 74 |
| M. pulmonis | MP-pul1 | AATTTTTGATCCGAGTCATT | 75 |
| | MP-pul2 | CATTTTTCTATCAATAGTTAT | 76 |
| | MP-pul3 | TATGTGTATCTTGCCAATTAG | 77 |
| | MP-pul4 | TTCTATCTTTCAAAACAAATA | 78 |
| | MP-pul5 | TATAAATTAATATGATAACGT | 79 |
| | MP-pul6 | TCATCAAAATGTAAAATTTTT | 80 |
| | MP-pul7 | AAAAATAAAATAGATACCTTA | 81 |
| | MP-pul8 | AAATAAATTTCAACAATAGGA | 82 |
| M. salivarium | MP-sal1 | TAATGGATTTAATTTTCGTG | 83 |
| | MP-sal2 | TATCAAATCAATATAATATTT | 84 |
| M. cloacale | MP-clo1 | AGTACAATTCTCACTGTTATG | 85 |
| | MP-clo2 | TAGAATATTCAAGACATATAC | 86 |
| M. falconis | MP-fal1 | GAGTACAACTTCTGTTATG | 87 |
| | MP-fal2 | AGAATACAAAAATATAGACAA | 88 |
| | MP-fal3 | ATTGAAAAATTATTAATTAAT | 89 |
| M. hyosynoviae | MP-hyos1 | CTAGACTAAAGTTAATGGTAC | 90 |
| | MP-hyos2 | AATTATCAAATTAATATTTCA | 91 |
| M. muris | MP-mur1 | TATAGAAAACCCCCACATCA | 92 |
| | MP-mur2 | TATTAGAATATTTTAAATATT | 93 |
| | MP-mur3 | GATTATTACACCATATTAGAA | 94 |
| | MP-mur4 | TCAATAAACCTAAATAAAAAA | 95 |
| M. primatum | MP-pri1 | GTAGACATAACCCCAGCTA | 96 |
| | MP-pri2 | CAAACGTCTATCGCTTTTTAG | 97 |
| | MP-pri3 | TCATGGGCTTTTAATAGGGTC | 98 |
| | MP-pri4 | ACCCCAACTCCCATCAAAAAT | 99 |
| M. | MP-spe1 | TTCATCGAGATAGTCATTTTA | 100 |
| spermatophilum | MP-spe2 | CAAACATACATTCAAATTTT | 101 |
| | MP-spe3 | TTTTGACTGAATGTTATTAAC | 102 |
| | MP-spe4 | TTTGTTATGTGACTTTTATGG | 103 |
| | MP-spe5 | AAAACAAACAATCTATACAAT | 104 |
| M. synoviae | MP-syn1 | TTGGCTTGGGCTATTGTATT | 105 |
| | MP-syn2 | GCGGTTGTGTATCGCTTTTTT | 106 |
| | MP-syn3 | ACCTCTCTTAAAATTGTTCTT | 107 |
| | MP-syn4 | CCGAGTTTAAATTATTGAATA | 108 |
| | MP-syn5 | CATCATAACAACATAACAATA | 109 |
| M. pneumoniae | MP-pne1 | GTAAATTAAACCCAAATCCC | 110 |
| | MP-pne2 | ATCTTTAATAAAGATAAATAC | 111 |
| | MP-pne3 | CTAAACAAAACATCAAAATCC | 112 |
| | MP-pne4 | AAAGAACATTTCCGCTTCTTT | 113 |
| M. genitalium | MP-gen1 | CACCCCTTAATTTTTTCGG | 114 |
| | MP-gen2 | AATGGAGTTTTTATTTTTTATTTA | 115 |
| | MP-gen3 | CCCAAATCAATGTTTGGTCTC | 116 |
| | MP-gen4 | CAACTAACACACTTGGTCAGT | 117 |
| | MP-gen5 | AGAATGTTTTTGAACAGTTC | 118 |
| | MP-gen6 | TAGTTCCAAAAATAAATACCA | 119 |
| M. bovis | MP-bov1 | TATAACCAAAATTAAAAGACCTA | 120 |
| | MP-bov2 | GTCATGGCTTTTATTAATAGG | 121 |
| U. urealyticum | UP-ure1 | CATTAAGTTGTCAGTGAA | 122 |
| | UP-ure2 | TAATTTACGTACTAATAAGTG | 123 |
| | UP-ure3 | TTTATTAAAATCCATATGAAT | 124 |
| | UP-ure4 | AAGCCACTTTTTTAAAAATTT | 125 |
| | UP-ure5 | CCATAATAATTAATTTATTAT | 126 |
| | UP-ure6 | ATTATCAACAAATCTTTCTAA | 127 |
| A. laidlawii | AP-lai1 | AACACTTAGCACAAGATGAC | 128 |
| | AP-lai2 | CTTTCTAAGGAGAAAGGCTAA | 129 |
| | AP-lai3 | ATGACTACTAGTAAGTAGTAA | 130 |
| | AP-lai4 | GTAGTAATATTCTCTAAATTT | 131 |
| | AP-lai5 | TTAAAGTAATTTAAGTGTTTC | 132 |
| | AP-lai6 | TAAATGATGTCTGAAAAGAAA | 133 |

*Mixed Base°| Code Name
M: A + C, W: A + T, Y: C + T, R: A + G
K: G + T, V: G + A + C, N: A + G + C + T

### BRIEF DESCRIPTION OF THE DRAWINGS

FIGS. 1a to 1f show multiple sequence alignments of each ITS region of Mycoplasma and Ureaplasma for selecting genus-specific probes.

FIGS. 2a to 2c show multiple sequence alignments of each ITS region of Acholeplasma for selecting genus-specific probes.

FIG. 3 shows a result of PCR amplification using primer pairs which can amplify ITS target sequences of many Mycoplasma and its related strains

FIG. 4 shows a microarray comprising probes for detecting genotypes of Mycoplasma and its related strains as a set on a support.

FIGS. 5a to 5k show results of image analysis of specific hybridization reaction of each probes for detecting genotypes of Mycoplasma and its related strains and results of numerical analysis calculated from pixel intensity.

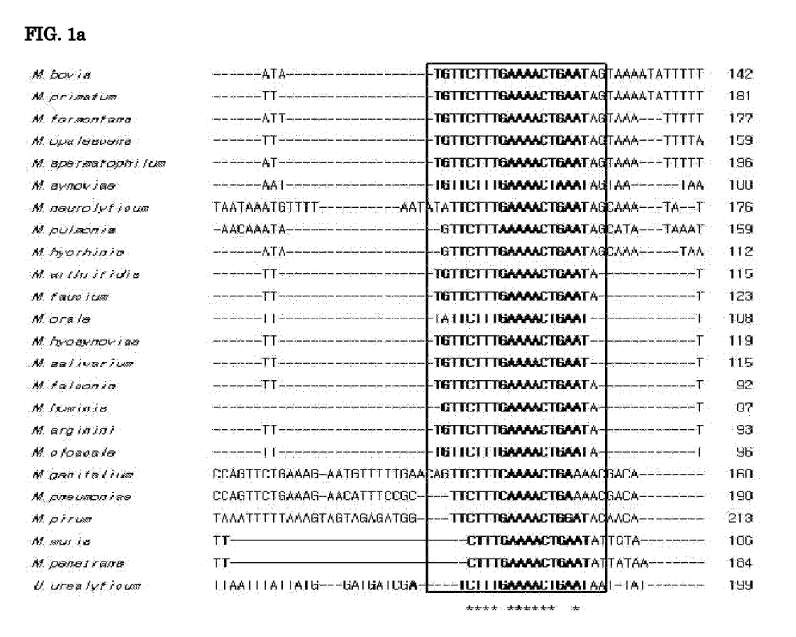

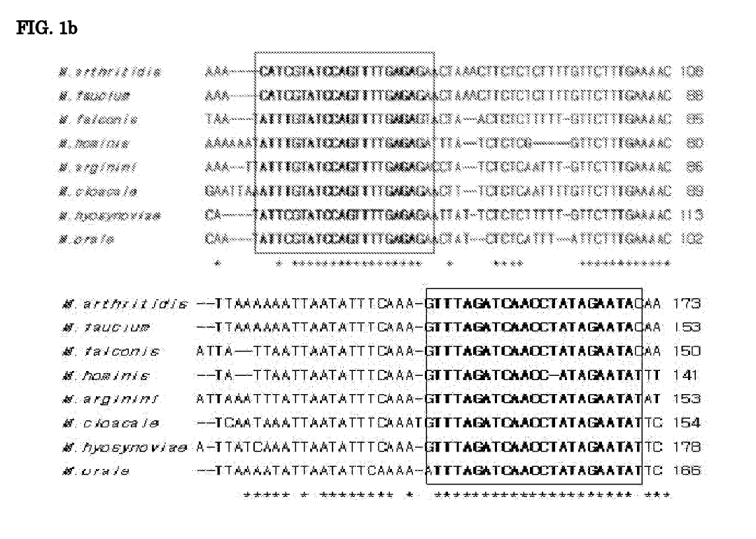

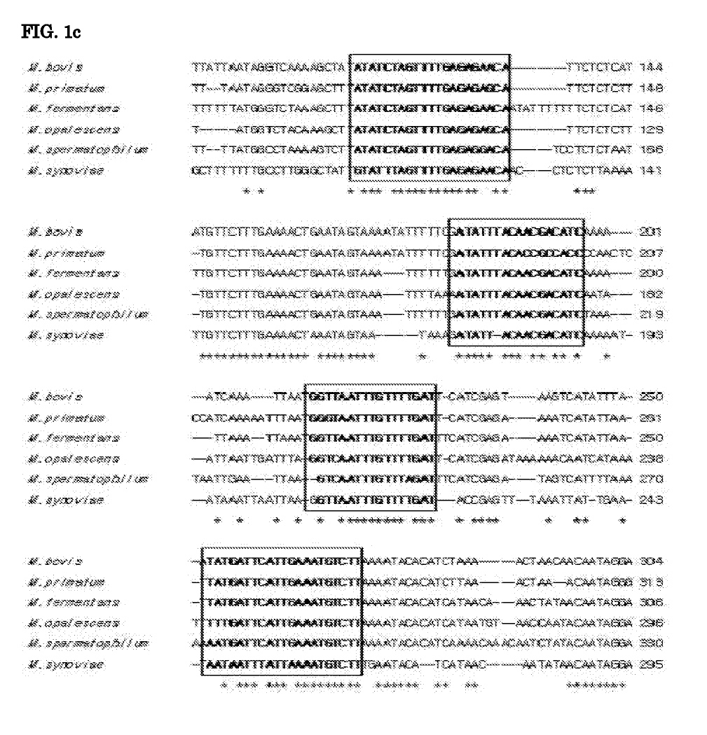

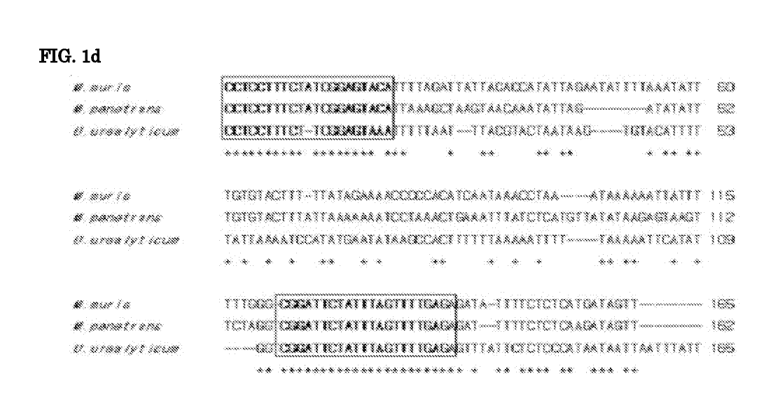

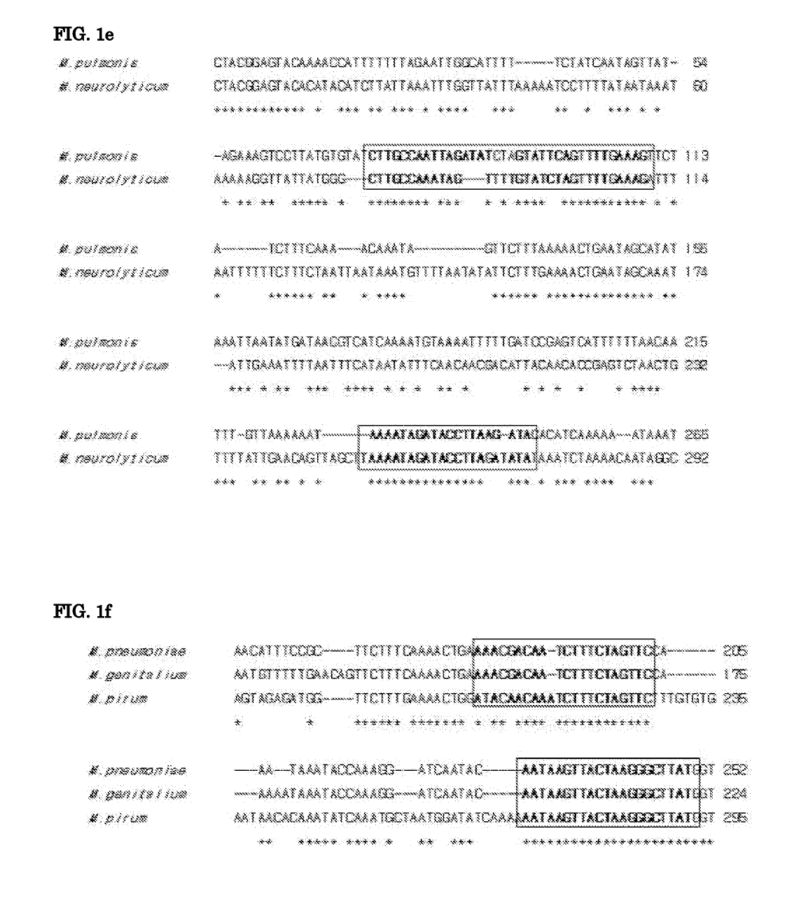

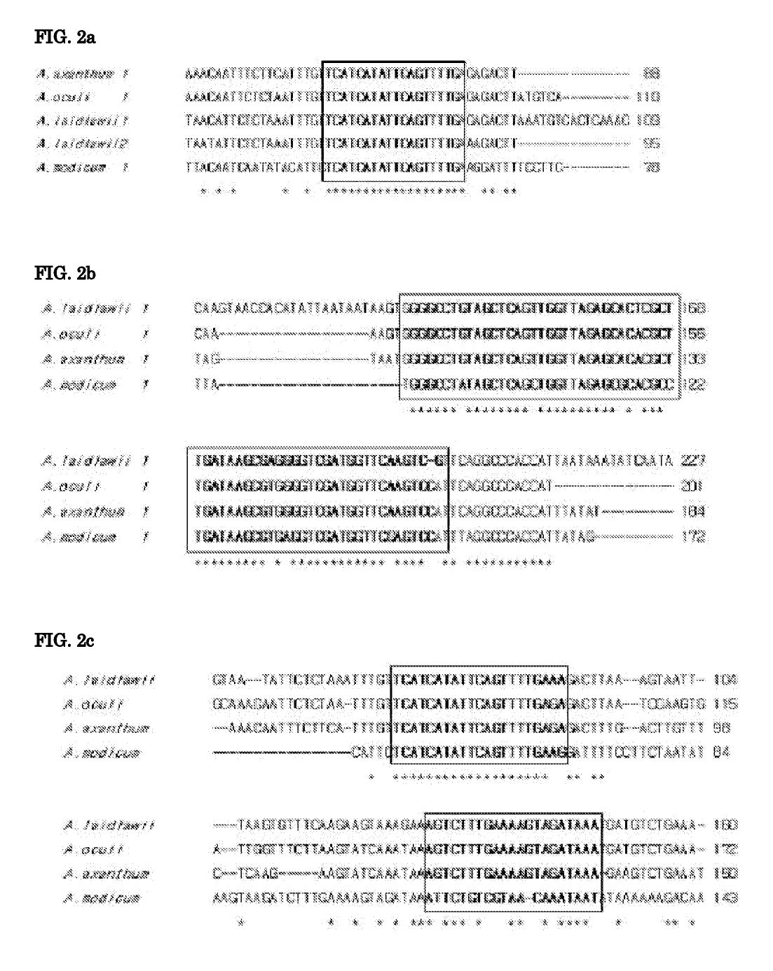

### BEST MODE FOR CARRYING OUT THE INVENTION

The present invention will be decribed in greater detail by means of following examples. The following examples are for illustrative purpose and are not intended to limit the scope of the invention.

### Example 1

### Incubation of Mycoplasma and its related strains and Isolation of Genomic DNA

Total 25 kinds of strains, including 1 kind of Acholeplasma, 23 kinds of Mycoplasma, and 1 kind of Ureaplasma were obtained from the American Type Culture Collection (ATCC). The strains were cultured in each culturing media under each culturing conditions according to manual provided by ATCC. From the cultured media, strain colonies were obtained with a white gold ear and input in 1.5 ml tube, 100 of InstaGene matrix (Bio-Rad, USA) was added thereto and suspended, and reaction was performed at 56° C. for 30 minutes in constant temperature bath. And then, the reactant was shook for 10 seconds, heated at 100° C. for 8 min, shook again for 10 sec, centrifuged at 12,000 rpm for 3 min, transferred to new tube, and freeze-stored at −20° C. The product was used as template DNA of PCR reaction.

The strains used were as followed:

Acholeplasma laidlawii (ATCC 25937)

Mycoplasma arginini (ATCC 23838)

Mycoplasma arthritidis (ATCC 19611)

Mycoplasma bovis (ATCC 27368)

Mycoplasma cloacale (ATCC 35276)

Mycoplasma falconis (ATCC 51372)

Mycoplasma faucium (ATCC 25293)

Mycoplasma fermentans (ATCC 19989)

Mycoplasma genitalium (ATCC 33530)

Mycoplasma hominis (ATCC 23114)

Mycoplasma hyorhinis (ATCC 17981)

Mycoplasma hyosynoviae (ATCC 25591)

Mycoplasma muris (ATCC 33757)

Mycoplasma neurolyticum (ATCC 19988)

Mycoplasma opalescens (ATCC 27921)

Mycoplasma orale (ATCC 23714)

Mycoplasma penetrans (ATCC 55252)

Mycoplasma pirum (ATCC 25960)

Mycoplasma pneumoniae (ATCC 15531)

Mycoplasma primatum (ATCC 15497)

Mycoplasma pulmonis (ATCC 14267)

Mycoplasma salivarium (ATCC 23064)

Mycoplasma spermatophilum (ATCC 49695)

Mycoplasma synoviae (ATCC 25204)

Ureaplasma urealyticum (ATCC 27618)

### Example 2

### Preparation of Probes for Detection of Mycoplasma and its Related Strains

The probes used for detection of Mycoplasma and its related strains were selected based on a result of multiple alignment of ITS sequences of Mycoplasma, Acholeplasma and Ureaplasma. Among Mycoplasma and its related species, 16S rRNA sequences has high similarity of 74-97%, whereas ITS sequences has lower similarity of 25.4-78.8% except for between M. salivarium and M. hyosynoviae, and M. hominis and M. falconis. In other words, ITS contains a region more polymorphic than 16S rRNA which is useful for designing probes for detection of Mycoplasma and its related strains. However, to complement specificity between M. salivarium and M. hyosynoviae, and M. hominis and M. falconis having a high ITS similarity, more restrictive and strict probes were designed.

In the present invention, the oligonucleotide probes for detection of Mycoplasma and its related strains were prepared by synthesizing 15-25 bases of specific probe with 15 bases of dT spacer at 5′ end. Probes for detection of Mycoplasma and its related strains are not restricted to the sequences disclosed in Tables 2 and 3 and any primer and probes comprising the sequences can be used in the present invention.

1. Preparation of Probes for Detection of Mycoplasma and Ureaplasma

\textcircled{1} Preparation of probes for genus-specific detection of Mycoplasma and Ureaplasma

For genus-specific hybridization with all Mycoplasma and Ureaplasma genus, probes of SEQ ID Nos. 7 and 8 in Table 2 were designed from conserved sequences of ITS of Mycoplasma. Further, each Group-based conserved sequences targeted to Mycoplasma ITS were designed as follows. For detecting Group I (M. arginins. M. arthritidis, M. cloacale, M. falconis, M. faucium, M. hominis, M. hyosynoviae, M. orale, M. salivarium), probes of SEQ ID Nos. 9 and 10 were designed. For detecting Group II (M. bovis. M. fermentans, M. opalescens, M. primatum, M. spermatophilum, M. synoviae), probes of SEQ ID Nos. 11, 12, 13 and 14 were designed. For detecting Group III (M. muris, M. penetrans, U. urealyticum), probes of SEQ ID Nos. 15 and 16 were designed. For detecting Group IV (M. neurolyticum, M. pulmonis), probes of SEQ ID Nos. 17, 18 and 19 were designed. For detecting Group V (M. genitalium, M. pirum, M. pneumoniae), probes of SEQ ID Nos. 20 and 21 were designed.

\textcircled{2} Preparation of Probes for Species-Specific Detection of Mycoplasma and Ureaplasma

For species-specific hybridization with each Mycoplasma and Ureaplasma species, 100 kind of probes of SEQ ID Nos. 28 to 127 in Table 3 were designed from species-specific sequences of ITS of Mycoplasma and Ureaplasma, which can detect 25 kind of Mycoplasma strains.

2. Preparation of Probes for Detection Acholeplasma

\textcircled{1} Preparation of Genus-Specific Probes for Detection Acholeplasma

For genus-specific hybridization with all Acholeplasma genus, probes of SEQ ID No. 22 in Table 2 was designed from conserved sequences targeted to both of ITS1 and ITS2 of Acholeplasma. Further, each Group-based conserved sequences targeted to each Acholeplasma ITS1 and ITS2 were designed as follows. For Group I targeted to ITS1, probes of SEQ ID Nos. 23, 24 and 25 were designed. For Group II targeted to ITS2, probes of SEQ ID Nos. 26 and 27 were designed.

\textcircled{2} Preparation of Species-Specific Probes for Detection Acholeplasma

For species-specific hybridization with each Acholeplasma species, probes of SEQ ID Nos. 128 to 133 in Table 3 were designed from species-specific sequences of ITS of Acholeplasma.

### Example 3

### Preparation of Target DNA

1. Preparation of Target DNA for Detection of Mycoplasma and its Related Strains

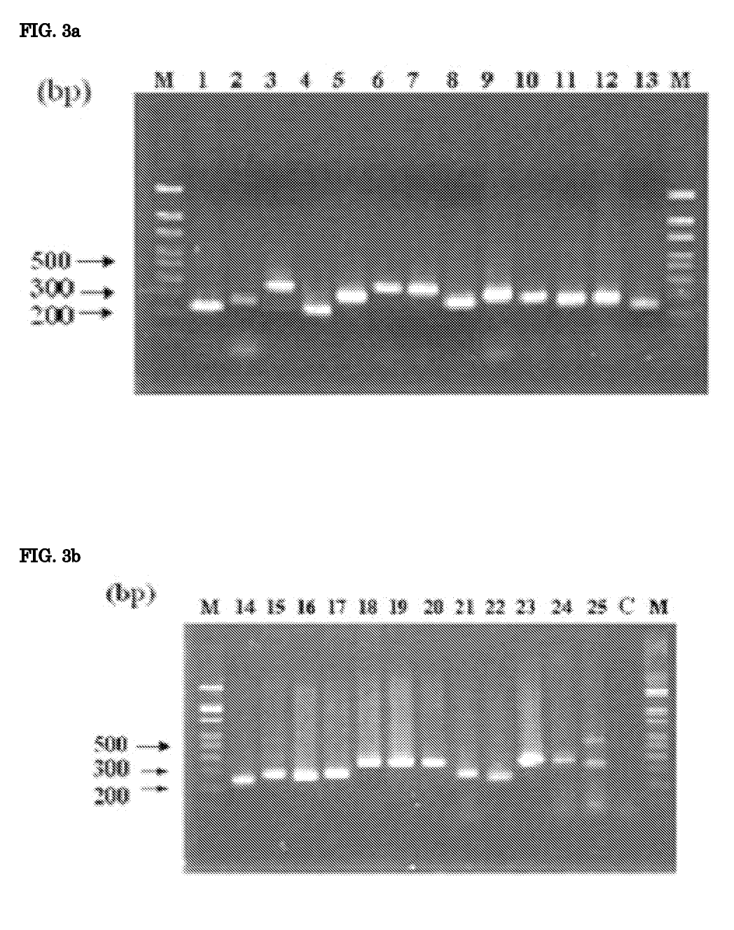

For preparing target DNA for detection of Mycoplasma and its related strains, 187˜290 bp size of ITS regions were selectively amplified using 5′-biotin-GTG(C/G)GG(A/C)TGGATCACCTCCT-3′ (MP16SF-2) and 5′-biotin-GCATCCACCA(A/T)A(A/T)AC(C/T)CTT-3′ (MP23SR-2), and 5′-biotin-AAAGTGGGCAATACCCAACGC-3′ (M78) and 5′-biotin-CCACTGTGTGCCCTTTGTTCCT-3′ (R34) which were biotin-labeled respectively (Tang et al., 2000.). To prepare genomic DNAs of Mycoplasma and its related strains isolated in Example 1, PCR were carried out using the above primers in the following conditions: denaturation at 94° C. for 3 minutes, 30 cycles of amplification at 94° C. for 30 seconds, at 55° C. for 2 minutes and at 72° C. for 2 minutes, and final extension at 72° C. for 10 minutes. After the reaction, the reaction products were analyzed by ELECTROPHORESIS on a 2% agarose gel. FIG. 3 is an electrophoresis image taken after the PCR performed using primers capable to amplify ITS target sequences of several Mycoplasma.

### Example 4

### Probe Immobilization on Support

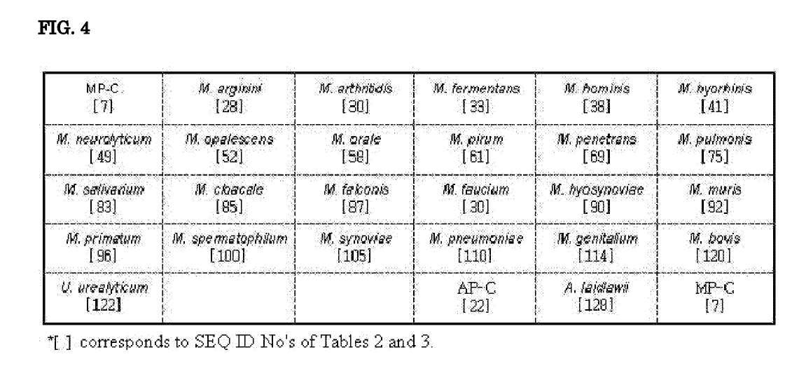

Among the probes prepared in Example 2, each representative probes for Mycoplasma, Acholeplasma and Ureaplasma were selected. Each of the selected probes was transferred to 384-well microplate, diluted to a concentration of 50 pmole by adding spotting solution, and immobilized on a slide glass using a microarrayer (Cartesian Technologies, USA). In FIG. 4, each probes for detection of Mycoplasma and its related strains correspond to SEQ ID Nos. 7, 28, 30, 33, 38, 41, 49, 52, 58, 61, 69, 75, 83, 85, 87, 30, 90, 92, 96, 100, 105, 110, 114, 120, 122, 22, 128, and 7 in order. Two spots of each kind of the probes were attached to the support and left in a slide box at room temperature for 24 hours or in a dry oven at 50° C. for about 5 hours to be fixed to the surface of the support.

### Example 5

### Unimmobilized Probe Washing

The slide glass after the process in Example 4 was washed with a 0.2% SDS buffer solution and then distilled water at room temperature to remove unimmobilized probes. The washed slide glass was immersed in a sodium borohydride (NaBH₄) solution for 5 minutes and then washed again at 100° C. Final washing with a 0.2% SDS solution and then distilled water was followed by centrifugation to fully dry the slide glass.

### Example 6

### Hybridization

The biotin-labeled target products prepared in Example 3 were thermally treated to be denaturated into single strands and cooled to 4° C. A hybridization reaction solution containing 2 μl of the target products was prepared. This hybridization reaction solution was portioned on the slide glass after the process in Examples 4 and 5, and the slide glass was covered with a cover slip and reacted at 25° C. for 1 hours.

### Example 7

### Unhybridized Tart DNA Washing

TO WASH OUT UNHYBRIDIZED TARGET DNAS, THE COVER SLIP WAS REMOVED USING A 2×SSC WASHING SOLUTION (300 MM NACL, 30 MM NA-CITRATE, PH 7.0), AND THE SLIDE WAS WASHED WITH 2×SSC AND THEN 0.2×SSC, FOLLOWED BY CENTRIFUGATION TO FULLY DRY THE SLIDE GLASS.

### Example 8

### Staining and Result Analysis

To determine hybridization of PCR products and probes, Cy5-streptavidin or Cy3-streptavidin (Amersham pharmacia biotech, USA) was diluted with 6×SSC and BSA (Bovine Serum Albumin), about 40 μl of dilutes was portioned on slide glass, and the slide glass was covered with a cover slip to block light and reacted at 50° C. for about 20 minutes. After the reaction, the cover slip was removed using a 2×SSC solution, and the slide was washed with 2×SSC and then 0.2×SSC. The hybridized result was scanned using a non-confocal laser scanner (GenePix 4000A, Axon Instruments, U.S.A.) and analyzed by image analysis.

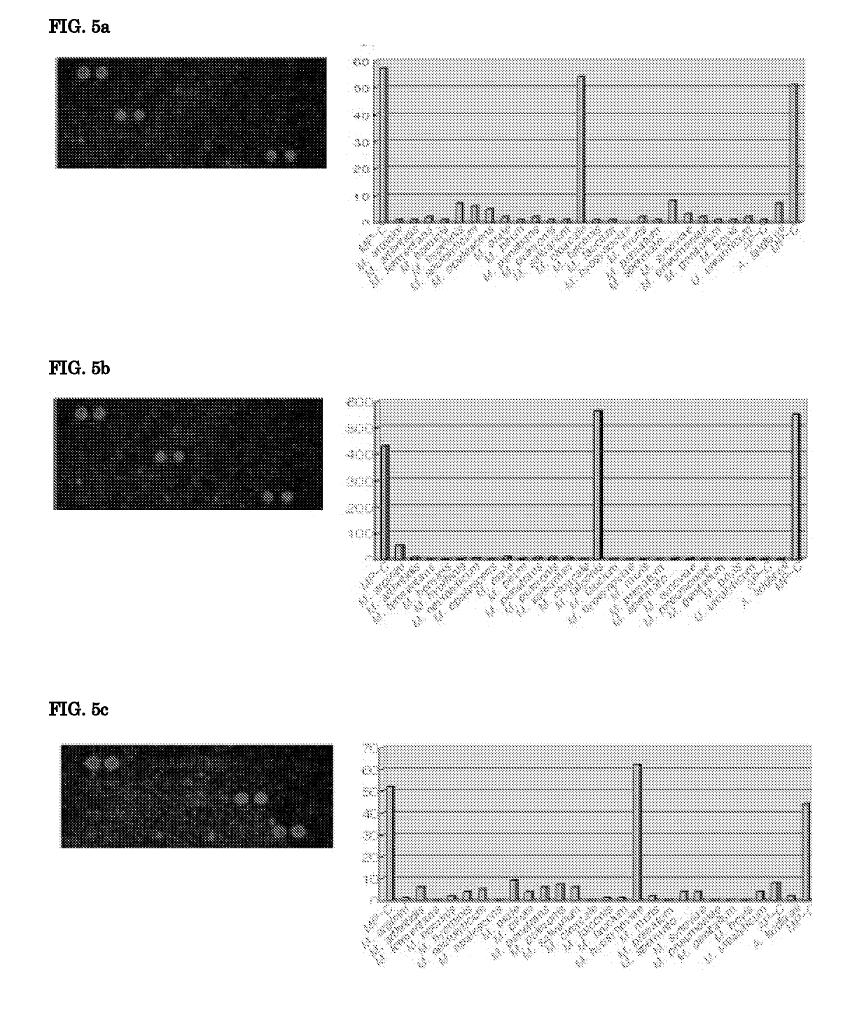

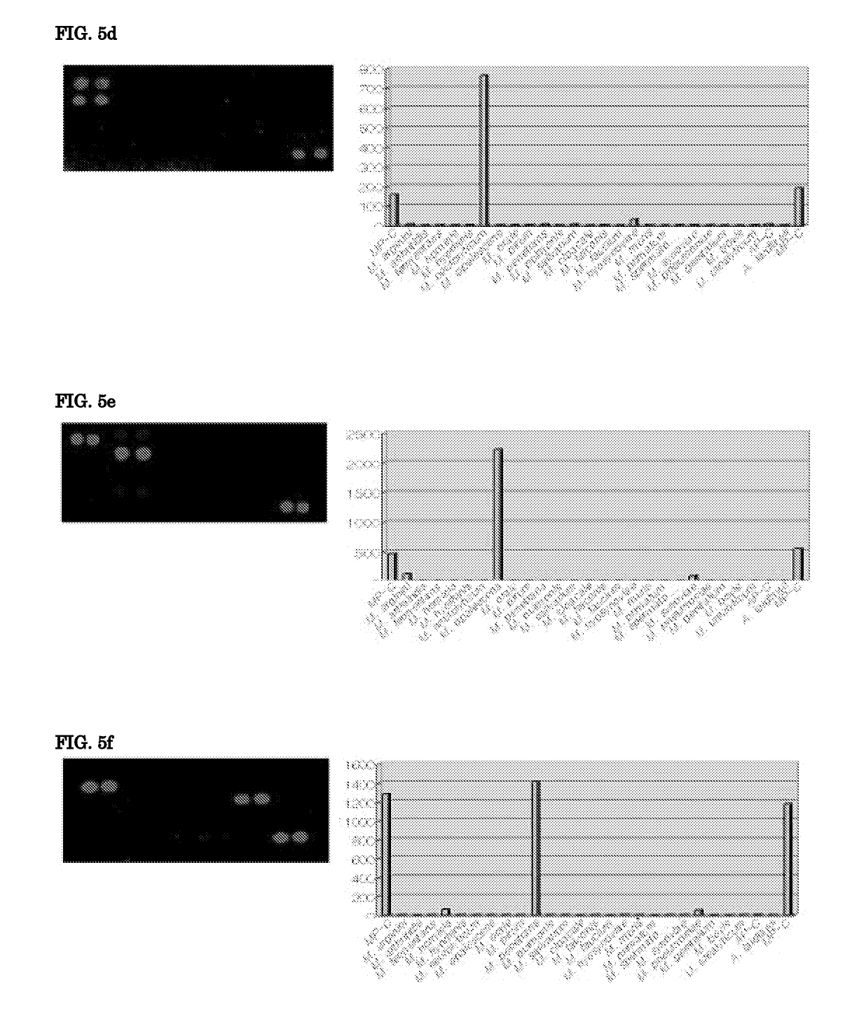

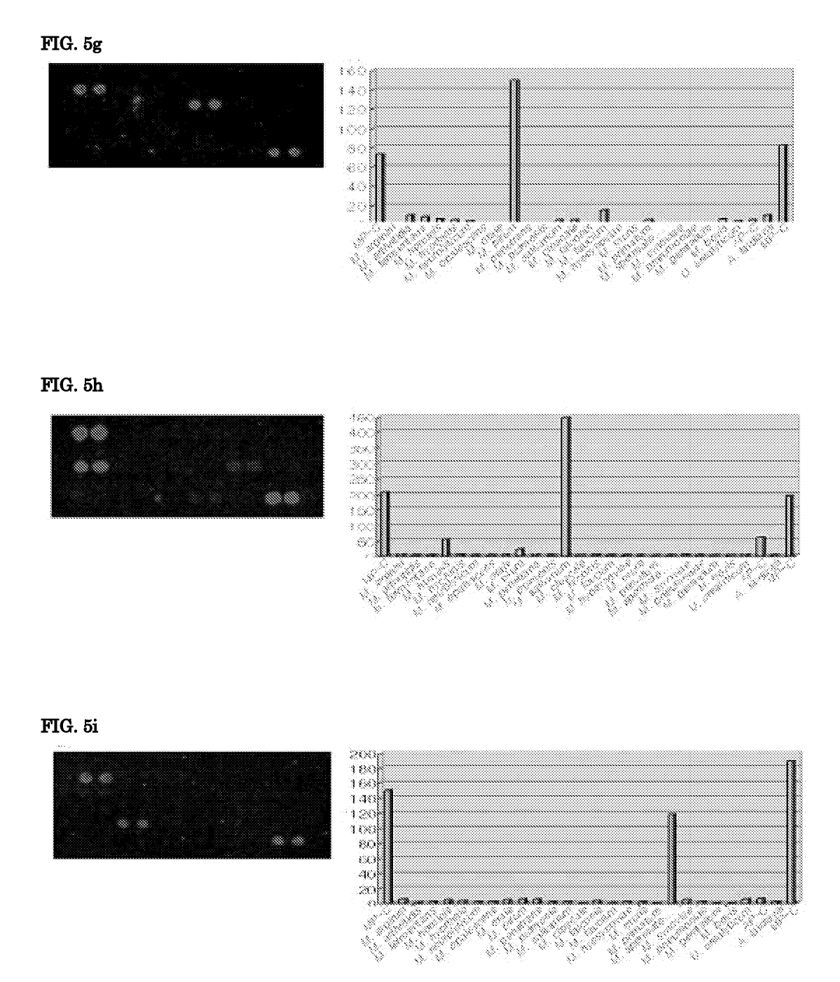

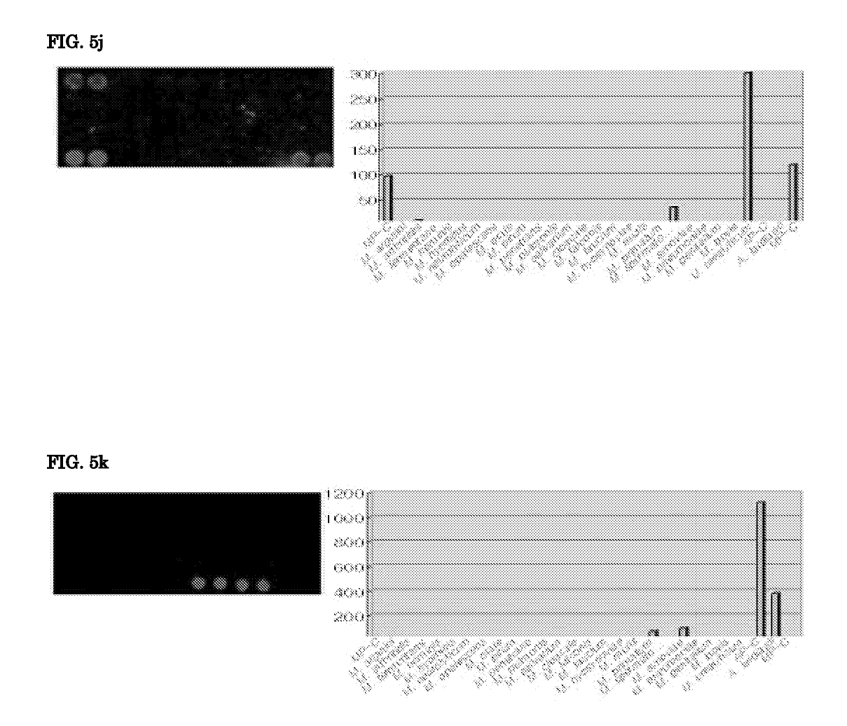

FIG. 5 shows results of image analysis of specific hybridization reaction of each probes for detecting genotypes of representative 11 kinds of Mycoplasma and its related strains and results of numerical analysis calculated from pixel intensity.

FIG. 5a shows results of hybridization reaction of genus-specific probe (SEQ ID No. 7) and species-specific probe (SEQ ID No. 85) of M. cloacale. FIG. 5b shows results of hybridization reaction of genus-specific probe (SEQ ID No. 7) and species-specific probe (SEQ ID No. 87) of M. falconis. FIG. 5c shows results of hybridization reaction of genus-specific probe (SEQ ID No. 7) and species-specific probe (SEQ ID No. 90) of M. hyosynoviae. FIG. 5d shows results of hybridization reaction of genus-specific probe (SEQ ID No. 7) and species-specific probe (SEQ ID No. 49) of M. neurolyticum. FIG. 5e shows results of hybridization reaction of genus-specific probe (SEQ ID No. 7) and species-specific probe (SEQ ID No. 52) of M. opalescens. FIG. 5f shows results of hybridization reaction of genus-specific probe (SEQ ID No. 7) and species-specific probe (SEQ ID No. 69) of M. penetrans. FIG. 5g shows results of hybridization reaction of genus-specific probe (SEQ ID No. 7) and species-specific probe (SEQ ID No. 61) of M. pirum. FIG. 5h shows results of hybridization reaction of genus-specific probe (SEQ ID No. 7) and species-specific probe (SEQ ID No. 83) of M. salivarium. FIG. 5i shows results of hybridization reaction of genus-specific probe (SEQ ID No. 7) and species-specific probe (SEQ ID No. 100) of M. spermatophilum. FIG. 5j shows results of hybridization reaction of genus-specific probe (SEQ ID No. 7) and species-specific probe (SEQ ID No. 122) of U. urealyticum. FIG. 5k shows results of hybridization reaction of genus-specific probe (SEQ ID No. 22) and species-specific probe (SEQ ID No. 128) of A. laidlawii.

### INDUSTRIAL APPLICABILITY

As described above, the present invention provides a rapid and accurate assay method capable of simultaneously detecting many Mycoplasma and its related strains from a single sample using a microarray comprising novel oligonucleotides for detecting Mycoplasma and its related strains which are known as a source of contamination of cell lines and biological products and human pathogenic.

Also, the present invention provides an objective and credible assay method capable of tracing a contamination source for preventing expansion of infective Mycoplasma and its related strains and controlling a contamination of Mycoplasma against biological products and stem cells or cord blood cells which are useful for gene therapy and cell therapy.

Further, the present invention provides very specific and sensitive hybridization assay for detecting Mycoplasma and its related strains using oligonucleotide probes designed based on sequence analysis of ITS region of many Mycoplasma Strains.

### REFERENCES

1. Doblhoff-Dier O, Collins C H. (2001) Biosafety: future priorities for research in health care. J Biotechnol. 85: 227-39.
2. Jung H, Wang S Y, Yang I W, Hsuch D W, Yang W J, Wang T H, Wang H S. (2003) Detection and treatment of Mycoplasma contamination in cultured cells. Chang Gung Med J. 26: 250-8.
3. Wisher M. (2002) Biosafety and product release testing issues relevant to replication-competent oncolytic viruses, Review. Cancer Gene Ther. 9: 1056-61.
4. Hopert A, Uphoff C C, Wirth H, Hauser H, Drexler H G. (1993) Specificity and sensitivity of polymerase chain reaction in complarision with other methods for the detection of Mycoplasma contamination in cell lines. J Immunol Methods. 164: 91-100.
5. Kong F, James G, Gordon S, Zelyski A, Gilbert G L. (2001) Species-Specific PCR for Identification of Common Contaminant Mollicutes in Cell Culture. Appl Environ Microbiol. 67: 3195-200.
6. Dorigo-zetsma J. W., Zaat S A J, Wertheim-van D, Spanjaard P M E, Rijntjes J, Waveren V, Jensen J S, Angulo A F, Dankert J. (1997) Comparision of PCR, culture, and serological tests for diagnosis of Mycoplasma pneumoniae respiratory tract infection in children. J Clin Microbiol. 37: 14-7.
7. Jensen J S, Borre M B, Dohn B. (2003) Detection of Mycoplasma genitalium by PCR Amplification of the 16S rRNA Gene. J Clin Microbiol. 41: 261-266.
8. Uphoff C C, Drexler H G. (2002) ComparativePCR analysis for detection of Mycoplasma infections in continuous cell lines. In Vitro Cell Dev Anim. 38: 79-85.
9. Gohlman H W, Weiner J 3rd, Schon A, Herrmann R. (2000) Identification of a small RNA within the pdh gene cluster of Mycoplasma pneumoniae and Mycoplasma genitalium. J. Bacteriol. 182: 3281-4.
10. Tang J, Hu M, Lee S, Roblin R. (2000) A polymerase chain reaction based method for detectiong Mycoplasma/Acholeplasma contaminants in cell culture. J Microbiol Methods. 39: 121-6.

What is claimed is:

1. An oligonucleotide for genus-specific genotyping of Mycoplasma and Ureaplasma strains, comprising any one sequence selected from SEQ ID Nos. 7 to 21 or its complementary sequence.

2. An oligonucleotide for species-specific genotyping of Mycoplasma and Ureaplasma strains, comprising any one sequence selected from SEQ ID Nos. 28 to 127 or its complementary sequence.

3. An oligonucleotide for genus-specific genotyping of Acholeplasma strains, comprising any one sequence selected from SEQ ID Nos. 22 to 27 or its complementary sequence.

4. An oligonucleotide for species-specific genotyping of Acholeplasma strains, comprising any one sequence selected from SEQ ID Nos. 128 to 133 or its complementary sequence.

5. A microarray comprising more than one oligonucleotide selected from genus-specific and species-specific oligonucleotides for genotyping Mycoplasma, Acholeplasma and Ureaplasma strains according to any one from claims 1 to 4 as probes attached on a support.

6. The microarray according to claim 5, wherein the probes are any one selected from a group consisting of DNA, RNA, PNA, LNA and HNA.

7. The microarray according to claim 5, wherein the support is any one selected from a group consisting of slide glass, plastic, membrane, semiconductive chip, silicon and gel.

8. A method for detecting Mycoplasma strains, comprising the following steps:
a) extracting nucleic acids from a sample;
b) amplifying target DNA among the extracted nucleic acids;
c) hybridizing the amplified target DNA with probes of a microarray according to claim 5; and
d) detecting signals generated from the hybridization reaction.

9. A kit for diagnosing Mycoplasma infection, comprising more than one oligonucleotide selected from genus-specific and species-specific oligonucleotides for genotyping Acholeplasma, Mycoplasma and Ureaplasma strains according to any one from claims 1 to 4.
